(19) United States
(12) Patent Application Publication
(10) Pub. No.: US 2008/0097338 A1
(43) Pub. Date: Apr. 24, 2008

(54) SAFETY SYRINGE WITH DISPOSABLE COMPONENTS AFTER USE

(75) Inventors: Wan Chang Cheng, Taipei (TW); Hsien Tsung Wang, Taipei (TW)

Correspondence Address: Kao H. Lu, 686 Lawson Ave., Havertown, PA, 19083, US

(73) Assignee: Wan Chang Cheng

(21) Appl. No.: 11/421,466

(22) Filed: May 31, 2006

(51) Int. Cl. A61M 5/24

(52) U.S. Cl. 604/201

(57) ABSTRACT

A syringe includes in one embodiment a fluid cartridge including a rear sliding member; a syringe barrel; a hypodermic needle assembly releasably secured to the syringe barrel; a nut member releasably secured to the syringe barrel; and a plunger including a rear handle, a front member disposed through the nut member to urge against a rear end of the sliding member, and an arm interconnected the handle and the front member. In response to dispensing fluid disengage the syringe barrel with the hypodermic needle assembly so as to separate the syringe into first and second portions, and wherein the first portion consists of the hypodermic needle assembly and the fluid cartridge and is adapted to discard, and the second portion consists of the nut, the plunger, and the syringe barrel and is adapted to sterilize for a next use.

### BACKGROUND OF THE INVENTION

1. Field of Invention

The present invention relates to syringes and more particularly to a safety syringe which, after use, some components thereof are discarded for eliminating the possibility of contamination, and the remaining components can be sterilized for future use for the purposes of saving cost and environmental protection.

2. Description of Related Art

Conventionally, a syringe for dispensing anesthetic is made of stainless steel and which is relatively high in cost. Thus, it is typical of sterilizing the used syringe rather than discarding it. However, in often times microorganisms may be not completely killed in the sterilization. As to the sterilization, it involves removing a needle from a syringe and sterilizing the syringe thereafter. However, it is highly possible that the needle may accidentally prick fingers of a medical worker and thereby contaminate the medical worker with microorganisms.

It is also typical for a syringe being made of plastics. Such syringes are usually discarded after use due to safety. Inevitably, however, such disposing may greatly increase operation cost of a hospital. This is also not desirable. Thus, it is desirable to provide a novel safety syringe having some recyclable components.

### SUMMARY OF THE INVENTION

It is therefore an object of the present invention to provide a safety syringe which, after use, some components thereof are discarded for eliminating the possibility of contamination, and the remaining components can be sterilized for future use for the purposes of saving cost and environmental protection.

In an aspect of the present invention there is provided a retractable syringe comprising a fluid cartridge including a rear sliding member; a syringe barrel; a front hypodermic needle assembly releasably secured to the syringe barrel; a nut member releasably secured to the syringe barrel; and a rear plunger including a rear handle, a front member disposed through the nut memberto urge against a rear end of the sliding member, and an arm interconnected the handle and the front member; wherein in response to dispensing fluid disengage the syringe barrel with the hypodermic needle assembly so as to separate the syringe into first and second portions, and wherein the first portion consists of the hypodermic needle assembly and the fluid cartridge and is adapted to discard, and the second portion consists of the nut, the plunger, and the syringe barrel and is adapted to sterilize for a next use.

In another aspect of the present invention there is provided a retractable syringe comprising a fluid cartridge including a rear sliding member; a syringe barrel and hypodermic needle assembly; a nut member releasably secured to the syringe barrel and hypodermic needle assembly; and a rear plunger including a rear handle, a front member disposed through the nut member to urge against a rear end of the sliding member, and an arm interconnected the handle and the front member; wherein in response to dispensing fluid disengage the syringe barrel and hypodermic needle assembly and the fluid cartridge with the nut member and the plunger, and wherein the syringe barrel and hypodermic needle assembly and the fluid cartridge are adapted to discard, and the nut member and the plunger are adapted to sterilize for a next use.

The above and other objects, features and advantages of the present invention will become apparent from the following detailed description taken with the accompanying drawings.

### BRIEF DESCRIPTION OF THE DRAWINGS

FIG. 1 is a perspective view of either first or second preferred embodiment of safety syringe according to the invention;

FIG. 2 is an exploded perspective view of a first configuration of a first preferred embodiment of safety syringe according to the invention;

FIG. 2A is a detailed view of the area in circle A of FIG. 2;

FIG. 2B is an exploded perspective view of a second configuration of the first preferred embodiment of safety syringe according to the invention;

FIG. 3 is an exploded perspective view of a second preferred embodiment of safety syringe according to the invention;

FIG. 3A is a detailed view of the area in circle B of FIG. 3;

FIG. 4 is a sectional view of the assembled syringe of FIG. 2 in a ready use state for fluid dispensing operation;

FIG. 5 is a view similar to FIG. 4 where fluid is being dispensed;

FIG. 6 is a sectional view showing the syringe being separated into two portions after use in which one portion can be discarded and the other portion can be sterilized for future use;

FIGS. 7 and 8 are sectional views showing the position of the projection prior to passing the lock member and the projection in its locked position after passing the lock member respectively;

FIGS. 9 and 10 are sectional views showing first and second configurations of the lock member respectively;

FIG. 11 is a perspective view of a third preferred embodiment of safety syringe according to the invention;

FIG. 12 is an exploded perspective view of the syringe shown in FIG. 11;

FIG. 13 is a sectional view of the assembled syringe of FIG. 11 in a ready use state for fluid dispensing operation;

FIG. 14 is a view similar to FIG. 13 where fluid is being dispensed;

FIG. 15 is a view similar to FIG. 13 where fluid has been dispensed and ready for second operation;

FIG. 16 is a view similar to FIG. 15 where fluid is being drawn into the empty syringe barrel from a medicine bottle prior to operating second fluid dispensing operation;

FIG. 17 is a sectional view of a fourth preferred embodiment of safety syringe according to the invention showing the syringe of being separated into two portions after use in which one portion can be discarded and the other portion can be sterilized for future use; and

FIG. 18 is an exploded perspective view of a fifth preferred embodiment of safety syringe according to the invention.

### DETAILED DESCRIPTION OF THE INVENTION

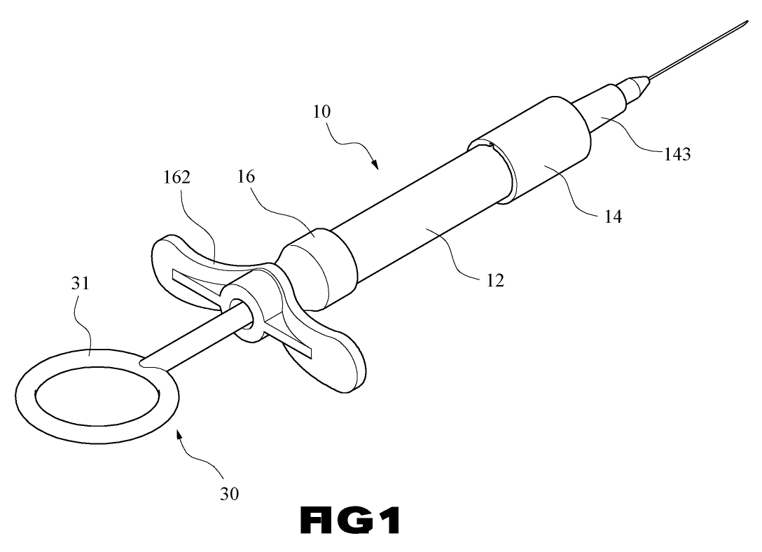

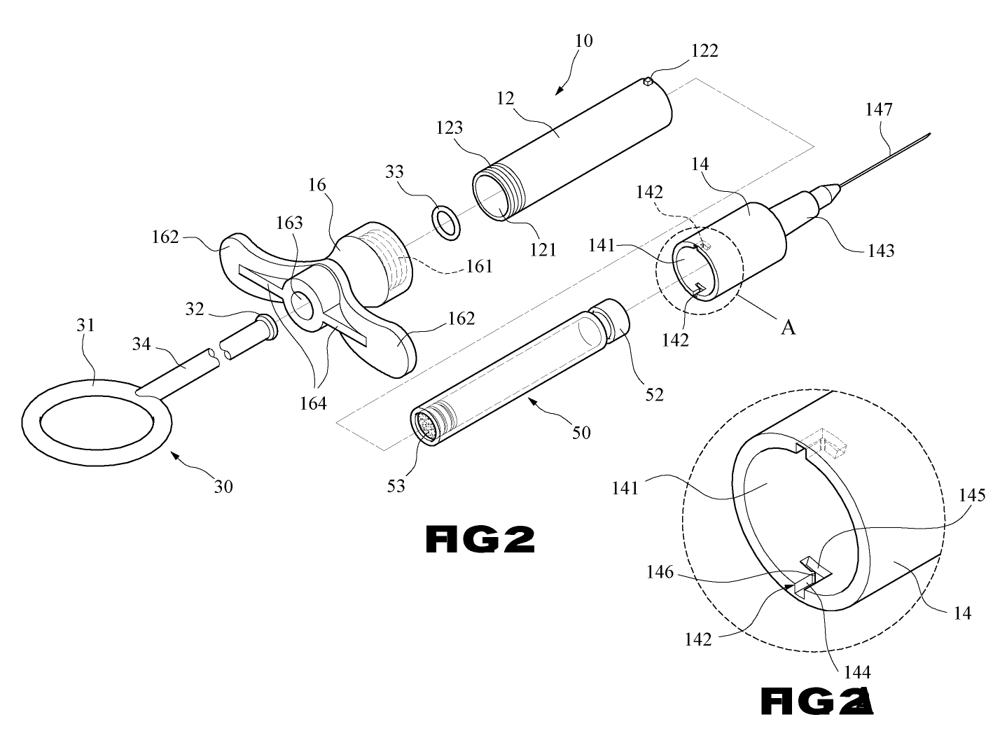

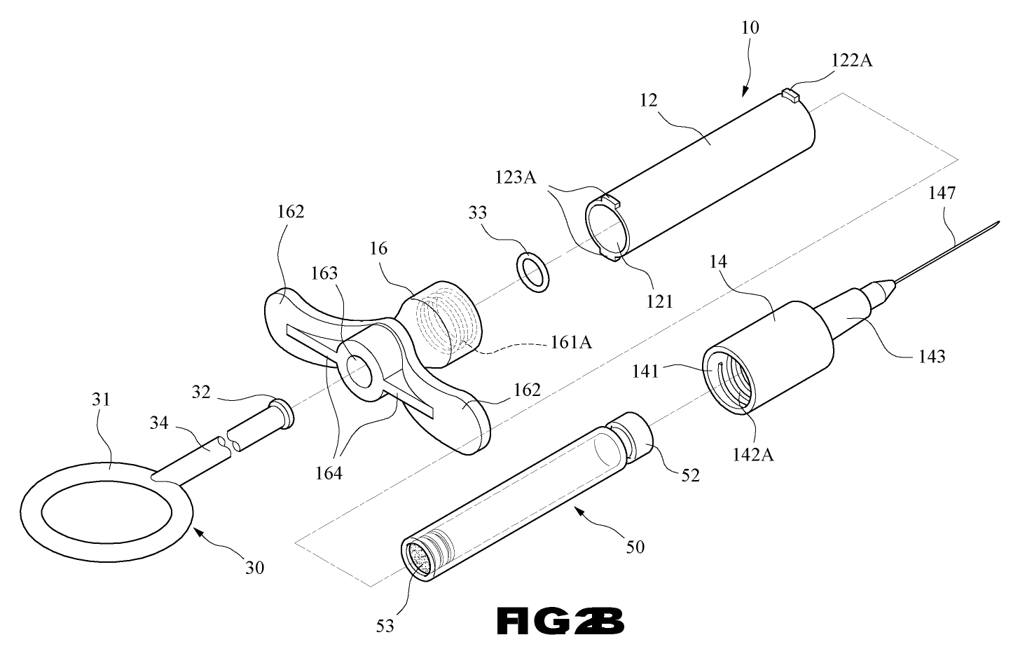

Referring to FIGS. 1, 2, 2A, and 2B, a safety syringe in accordance with a first preferred embodiment of the invention is shown. The syringe comprises a syringe barrel and hypodermic needle assembly 10, a plunger 30, and a fluid cartridge 50. Each component is discussed in detailed below.

The syringe barrel and hypodermic needle assembly 10 comprises an elongated, cylindrical syringe barrel 12, a hypodermic needle assembly 14, and a wing nut member 16.

In a first configuration of the embodiment (see FIGS. 2 and 2A) the syringe barrel 12 comprises two opposite projections 122 on an outer surface proximate its front end, an axial bore 121, and rear outer threads 123. The needle assembly 14 comprises a cylindrical rear housing 141, two rear opposite L-shaped grooves 142 adapted to secure to the projections 122 as detailed later, each groove 142 including an axial section 144, a transverse section 145, and a lock member 146 at a joining point of the axial and transverse sections 144 and 145, a conic nose 143 threadedly secured to and extended forward from the housing 141, and a needle 147 extended from inside of the housing 141 through the nose 143. The wing nut member 16 comprises front inner threads 161 adapted to secure to the outer threads 123, two rear wings 162, an axial bore 163, and two opposite braces 164 each interconnected a main cylindrical portion of the wing nut member 16 and the wing 162.

In a second configuration of the embodiment (see FIG. 2B) the syringe barrel 12 comprises two opposite, arcuate first protrusions 122A on an outer surface proximate its front end, an axial bore 121, and two opposite, arcuate second protrusions 123A on an outer surface proximate its rear end. The needle assembly 14 comprises a cylindrical rear housing 141, rear inner threads 142A adapted to secure to the first protrusions 122A as detailed later, a conic nose 143 threadedly secured to and extended forward from the housing 141, and a needle 147 extended from inside of the housing 141 through the nose 143. The wing nut member 16 comprises front inner threads 161A adapted to secure to the second protrusions 123A, two rear wings 162, an axial bore 163, and two opposite braces 164 each interconnected a main cylindrical portion of the wing nut member 16 and the wing 162.

The plunger 30 comprises a rear ring 31 as a handle for enabling a finger to insert into, a forward enlargement 32 having an annular trough and a diameter slightly smaller than that of the bore 163, a separate ring-shaped rubber seal 33 fastened on the trough of the enlargement 32, and an arm 34 interconnected the ring 31 and the enlargement 32. The enlargement 32 and the seal 33 are shaped to snugly insert through the bore 163 for fastening the plunger 30 and the wing nut member 16 together.

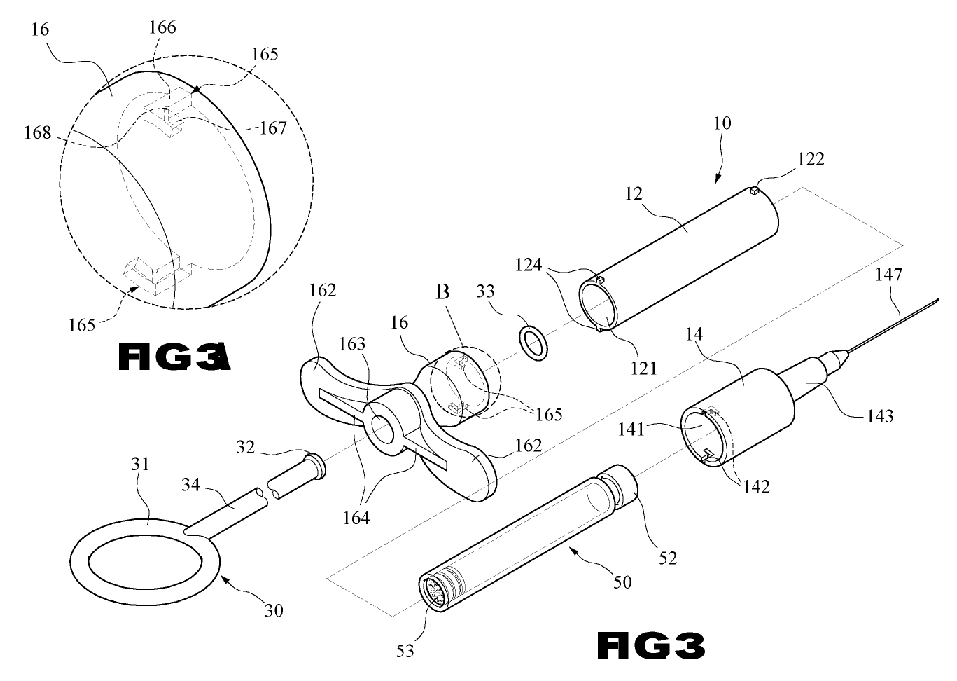

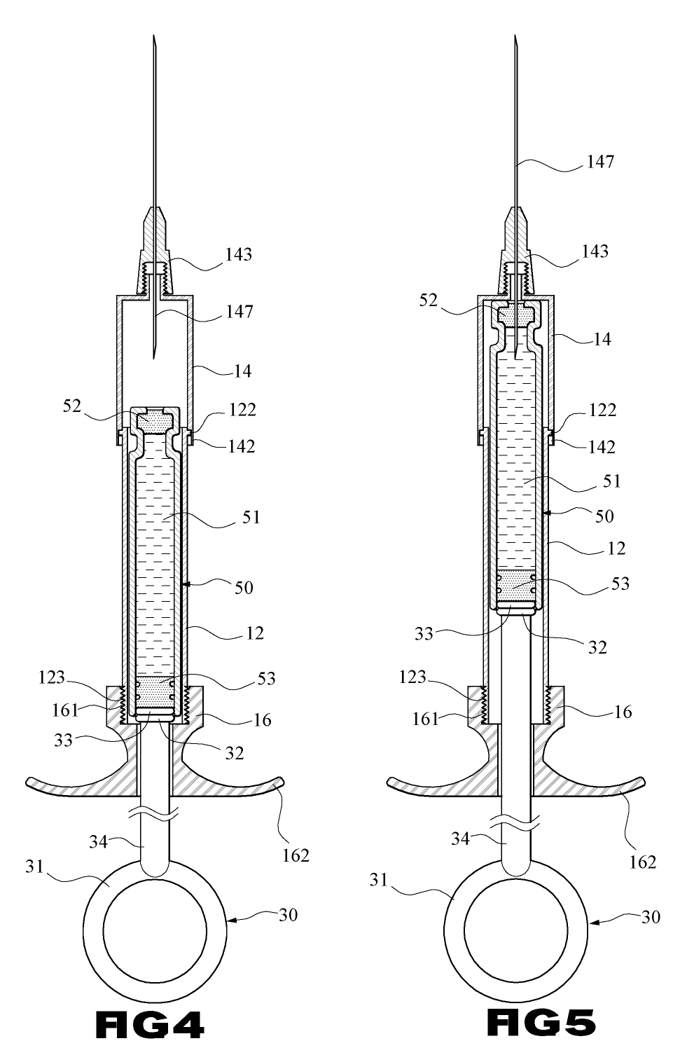

The fluid cartridge 50 is a cylindrical container made of glass and comprises a forward extension having a rubber member 52 (see FIGS. 4-5) fastened therein, and a cylindrical sliding member 53 shaped to fit snugly inside the hollow fluid cartridge 50 and slide therethrough from an initial position near the rear end in a fully charged state of the fluid cartridge 50 (i.e., fluid (e.g., anesthetic) 51 is contained in the fluid cartridge 50) toward the rubber member 52 as detailed later.

Referring to FIGS. 3 and 3A, a safety syringe in accordance with a second preferred embodiment of the invention is shown. The second embodiment is identical to the first configuration of the first embodiment, except that the rear outer threads 123 are replaced by two opposite second projections 124 on an outer surface proximate its rear end, and the front inner threads 161 are replaced by two opposite L-shaped second grooves 165 adapted to secure to the second projections 124 as detailed later, each second groove 165 including an axial section 165, a transverse section 167, and a lock member 168 at a joining point of the axial and transverse sections 165 and 167.

Referring to FIGS. 4, 5, and 6, further assembly and a fluid dispensing operation of the first configuration of the first embodiment will be described in detailed below. Insert the fluid cartridge 50 into the hollow syringe barrel 12. Next, attach the enlargement 32 to the sliding member 53. Next, secure the inner threads 161 onto the outer threads 123. As an end, the syringe barrel 12 and the wing nut member 16 are secured together (i.e., the syringe is assembled). For filling the fluid cartridge 50 with fluid 51, a medical worker may further insert the fluid cartridge 50 into the syringe barrel 12 until a front end of the fluid cartridge 50 is stopped by a bottom end of the needle assembly 14. At the same time, a rear pointed end of the needle 147 pierces through the rubber member 52 into the fluid cartridge 50. Next, slide the sliding member 53 to a position proximate the rear end of the needle 147 by holding and pushing the ring 31. Next, pierce a front pointed end of the needle 147 into a medical bottle and draw fluid 51 from the medical bottle into the fluid cartridge 50 by creating a vacuum in the fluid cartridge 50 by pulling the ring 31 until the fluid cartridge 50 is full of fluid 51. Finally, remove the needle 147 from the medical bottle and disengage the fluid cartridge 50 with the needle 147 by further pulling the ring 31 as shown in FIG. 4.

In a fluid dispensing operation, a medical worker may introduce the needle 147 into the skin of a patient to inject fluid 51 by slidably pushing the sliding member 53 by holding and pushing the ring 31 (see FIG. 5). After use, the medical worker may detach the projections 122 from the grooves 142. As such, the syringe is separated into two portions (see FIG. 6). One portion consists of the needle assembly 14 and the fluid cartridge 50 and the other portion consists of the plunger 30 and the syringe barrel 12. One portion is discarded for eliminating the possibility of contamination. Note that a cap (not shown) may be put on the needle 147 for safety prior to the discard. The other portion can be sterilized for future use for the purposes of saving cost and environmental protection.

Note that further assembly and a fluid dispensing operation of either the second configuration of the first embodiment or the second embodiment is generally identical to that described above with respect to the first configuration of the first embodiment. Accordingly, further description is omitted for purposes of brevity and convenience only, and is not limiting.

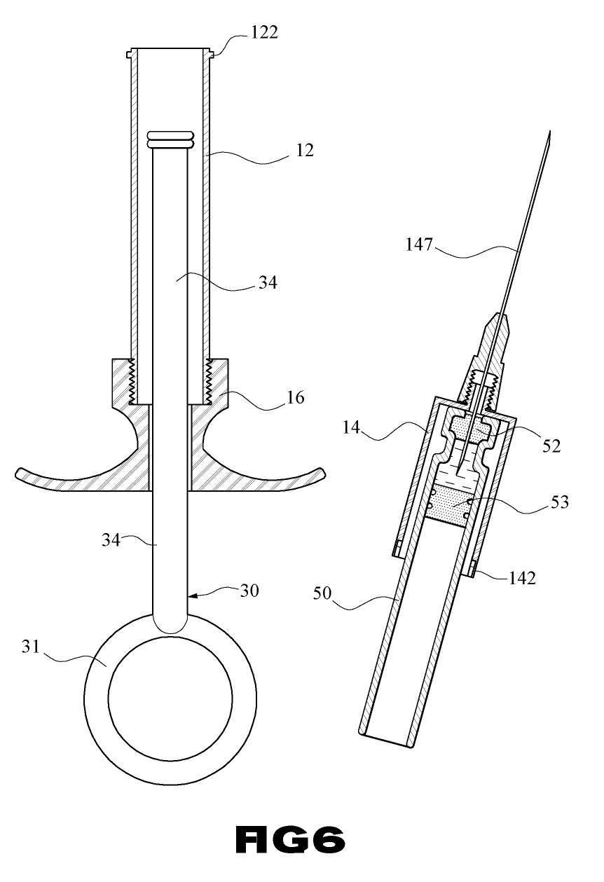

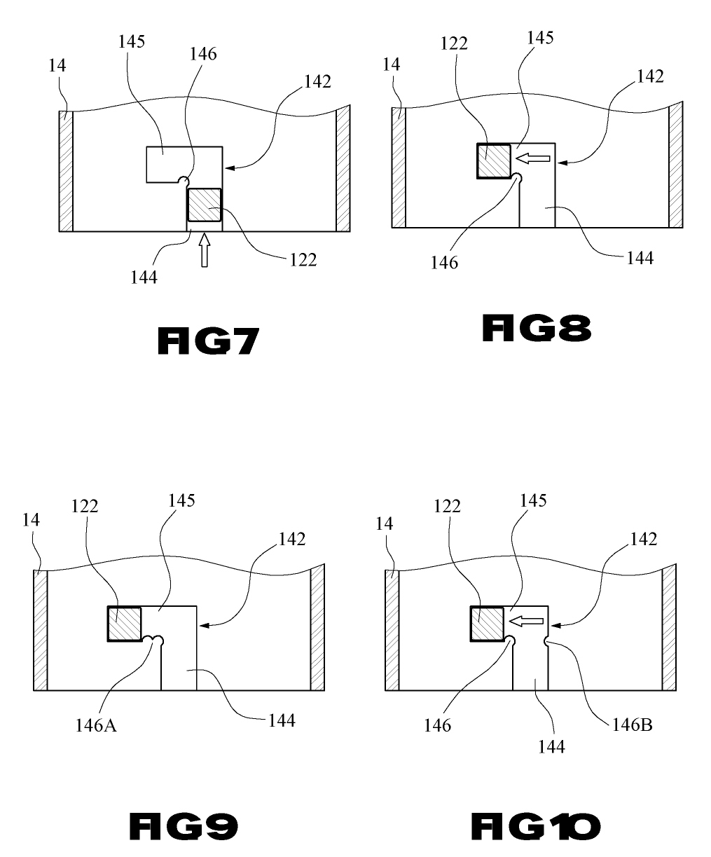

Referring to FIG. 7, the projection 122 is passing the axial section 144. Referring to FIG. 8, the projection 122 has passed the lock member 146 to fasten in a locked position in the transverse section 145. As a result, the syringe barrel 12 and the needle assembly 14 are secured together. For disengaging the syringe barrel 12 with the needle assembly 14 after using the syringe, the steps discussed with reference to FIGS. 7 and 8 are traversed in the opposite direction.

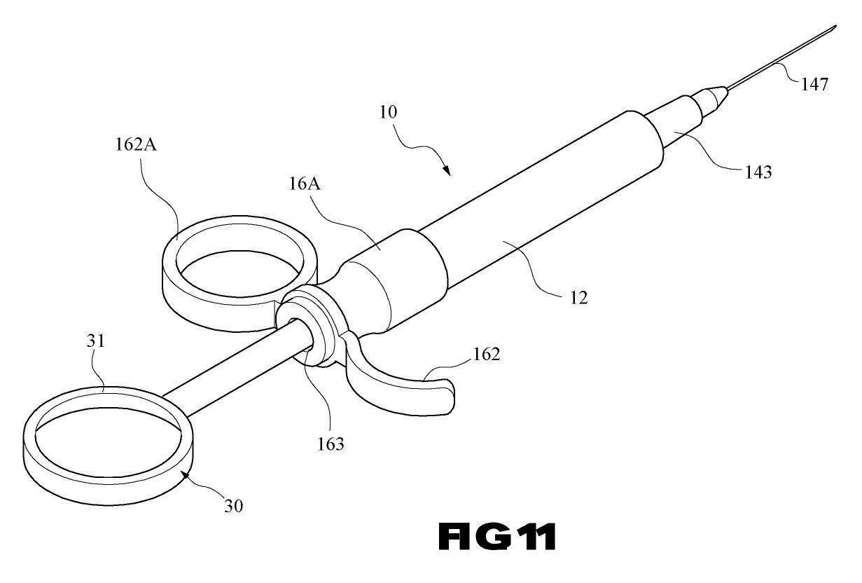

Referring to FIG. 9, in a first configuration the lock member 146 is implemented as two tabs 146A located side by side. Referring to FIG. 10, in a second configuration the lock member 146 is implemented as a tab 146 and an opposite raised member 146B to operate the same function as mentioned above.

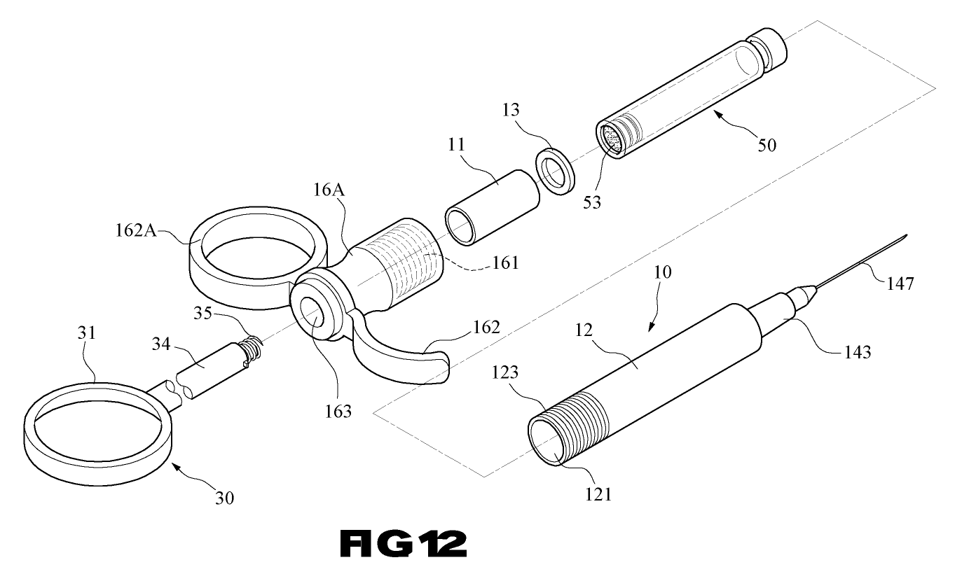

Referring to FIGS. 11 and 12, a safety syringe in accordance with a third preferred embodiment of the invention is shown. The third embodiment is identical to the first configuration of the first embodiment, except that the syringe barrel 12 and the housing of the needle assembly 14 are formed integrally without the provision of the projections 122 and the grooves 142, in order to perform a second fluid dispensing operation, the wing nut member 16A has changed to one rear wing 162 and one rear ring 162A for facilitating one finger holding in the ring 162A and the other finger pulling back the ring 31 of the plunger 30, the enlargement 32 is replaced by a helical spring 35 anchored on a front, cylindrical extension of the arm 34, a slip resistant ring 13 is engaged with a rear casing end of the fluid cartridge 50, and a cylinder 11 is further provided to engage with a rear end of the slip resistant ring 13. Both the slip resistant ring 13 and the cylinder 11 are disposed in a rear portion of the syringe barrel 12 to prevent the fluid cartridge 50 from being slipping (holding the fluid cartridge in place) when the cylinder 11 is forced to push on the rear casing end of the fluid cartridge 50 by the wing nut member 16A and a helical spring 35 is going to pierce into the rear of the sliding member 53 of the fluid cartridge 50 by rotating the ring 31 clockwise as shown in FIG. 15.

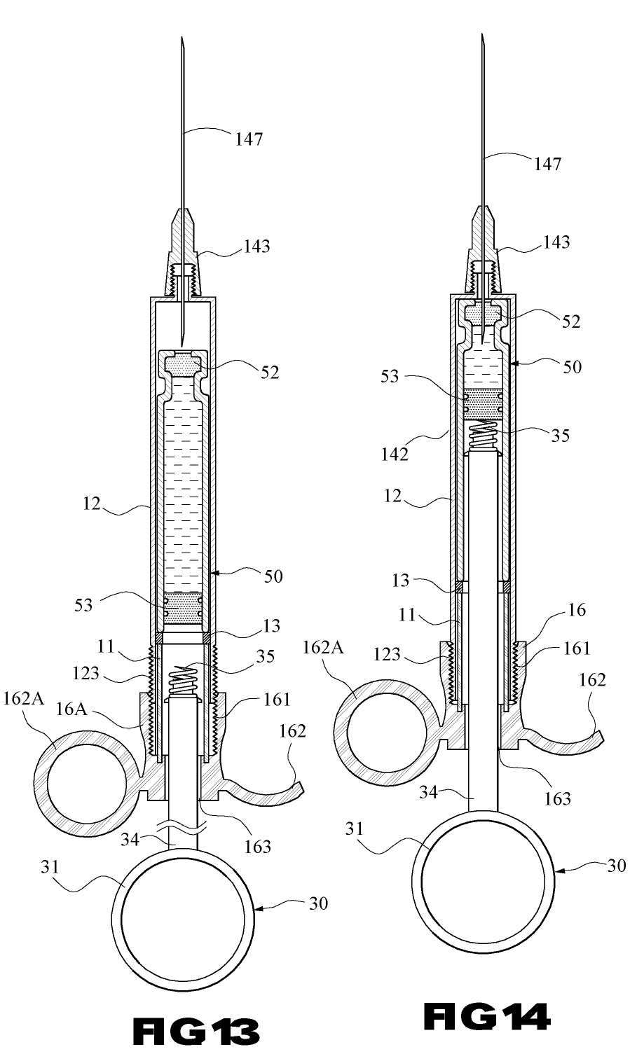

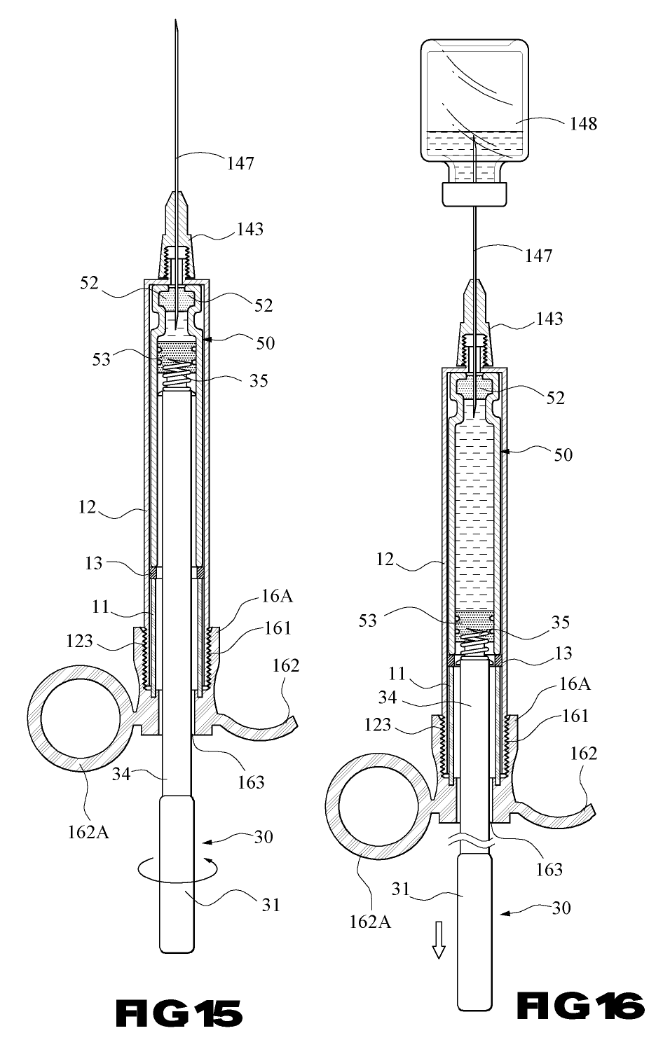

Referring to FIGS. 13 to 16, a fluid dispensing operation of the third embodiment will be described in detailed below. First, a medical worker may hold and push the ring 31 to cause the spring 35 to contact the sliding member 53 and further slide the fluid cartridge 50 forward until the front end of the fluid cartridge 50 is stopped by the bottom end of the needle assembly 14. At the same time, the rear pointed end of the needle 147 pierces through the rubber member 52 into the fluid cartridge 50 (see FIGS. 13 and 14). Next, the medical worker may introduce the needle 147 into the skin of a patient to inject fluid 51 by slidably pushing the sliding member 53.

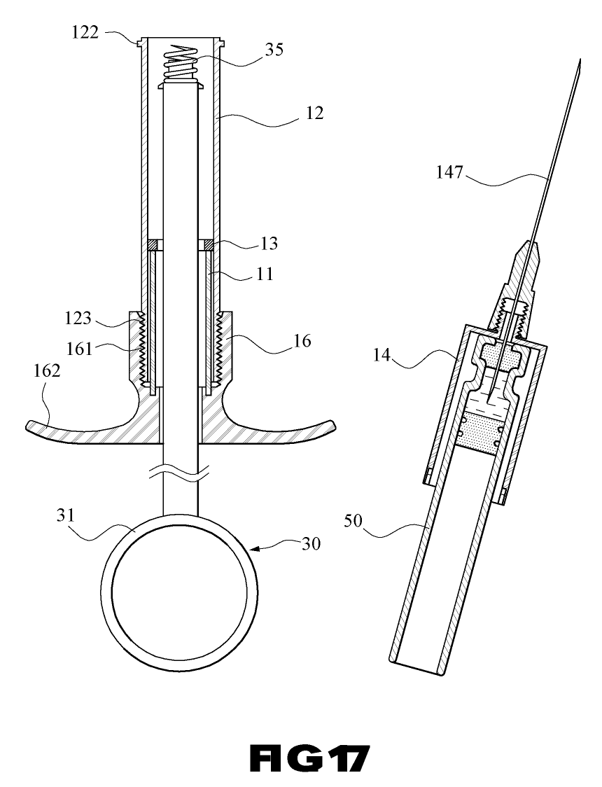

After finishing the first injection, the medical worker may rotate the wing nut member 16 clockwise to further holding the casing of the fluid cartridge 50 in place by urging the slip resistant ring 13 through the cylinder 11 against the rear casing end of the fluid cartridge 50 prior to piercing a pointed end of the spring 35 into the sliding member 53 by rotating the ring 31 clockwise (see FIG. 15). Next, the medical worker may pierce a front pointed end of the needle 147 into a medical bottle 148 (see FIG. 16) and draw fluid from the medical bottle 148 into the fluid cartridge 50 by creating a vacuum in the fluid cartridge 50 by pulling the ring 31 until the fluid cartridge 50 is full of fluid for the purpose of operation of second injection. This completes the fluid filling operation (see FIG. 16).

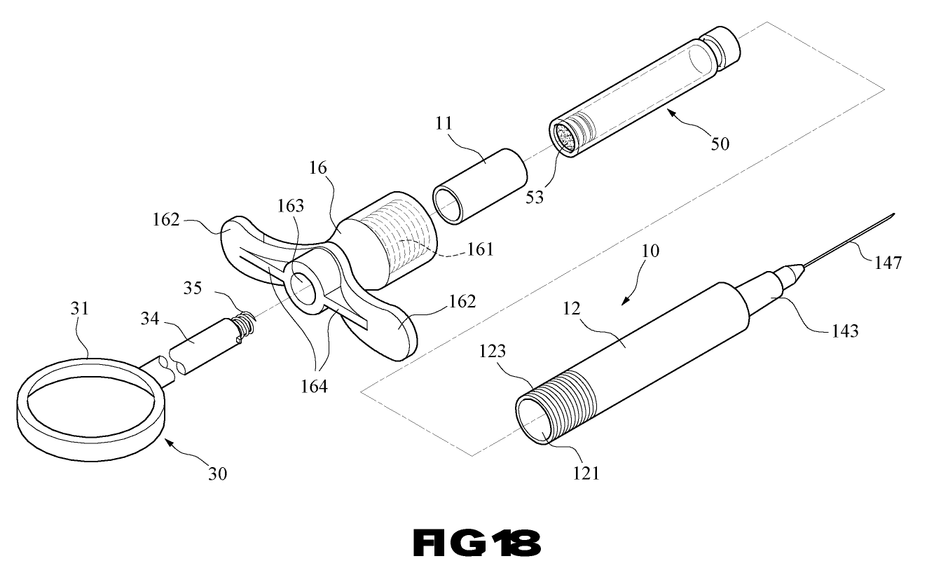

Referring to FIG. 17, a safety syringe in accordance with a fourth preferred embodiment of the invention is shown. According the embodiment, the integrally formed syringe barrel 12 and the housing of the hypodermic needle assembly 14 may be formed separately and are adapted to releasably secure together as the first configuration of the first embodiment. Thus, similar to the first configuration of the first embodiment, after use the medical worker may detach the projections 122 from the grooves 142. As such, the syringe is separated into two portions. One portion consists of the needle assembly 14 and the fluid cartridge 50 and the other portion consists of the plunger 30, the cylinder 11 and the syringe barrel 12. One portion is discarded for eliminating the possibility of contamination. Note that a cap (not shown) may be put on the needle 147 for safety prior to the discard. The other portion can be sterilized for future use for the purposes of saving cost and environmental protection.

Referring to FIG. 18, a safety syringe in accordance with a fifth preferred embodiment of the invention is shown. The fifth embodiment is identical to the third embodiment, except that the slip resistant ring 13 is eliminated since the cylinder 11 is formed of plastics or rubber having a slip resistant feature.

While the invention herein disclosed has been described by means of specific embodiments, numerous modifications and variations could be made thereto by those skilled in the art without departing from the scope and spirit of the invention set forth in the claims.

What is claimed is:

1. A retractable syringe comprising:
a fluid cartridge including a rear sliding member;
a syringe barrel;
a front hypodermic needle assembly releasably secured to the syringe barrel;
a nut member releasably secured to the syringe barrel; and
a rear plunger including a rear handle, a front member disposed through the nut member to urge against a rear end of the sliding member, and an arm interconnected the handle and the front member;
wherein in response to dispensing fluid disengage the syringe barrel with the hypodermic needle assembly so as to separate the syringe into first and second portions, and wherein the first portion consists of the hypodermic needle assembly and the fluid cartridge and is adapted to discard, and the second portion consists of the nut member, the plunger, and the syringe barrel and is adapted to sterilize for a next use.

2. The syringe of claim 1, wherein the syringe barrel comprises two opposite front projections on an outer surface and rear outer threads.

3. The syringe of claim 2, wherein the hypodermic needle assembly comprises a cylindrical rear housing and two rear opposite L-shaped grooves adapted to lockingly engage with the projections.

4. The syringe of claim 2, wherein the nut member comprises front inner threads adapted to secure to the outer threads.

5. The syringe of claim 1, wherein the syringe barrel comprises two opposite, arcuate, front first protrusions on an outer surface and two opposite, arcuate second protrusions on the outer surface.

6. The syringe of claim 5, wherein the hypodermic needle assembly comprises a cylindrical rear housing and rear inner threads adapted to lockingly engage with the first protrusions.

7. The syringe of claim 5, wherein the nut member comprises front inner threads adapted to secure to the second protrusions.

8. The syringe of claim 3, wherein the groove comprises an axial section, a transverse section, and a lock member at a joining point of the axial and transverse sections.

9. The syringe of claim 8, wherein the lock member is implemented as a tab, two tabs side by side, or a tab and an opposite raised member.

10. The syringe of claim 1, wherein the front member is an enlargement having an annular trough and a diameter slightly smaller than that of the nut, and the plunger further comprises a separate ring-shaped rubber seal fastened on the trough of the enlargement.

11. The syringe of claim 1, wherein the syringe barrel comprises a slip resistant ring engaged with a rear casing end of the fluid cartridge, and a cylinder engaged with a rear end of the slip resistant ring.

12. The syringe of claim 11, wherein the front member is a helical spring, and whereby in the fluid dispensing pushing the handle will cause the spring to pierce into the sliding member by pushing the cylinder and the slip resistant ring on the rear casing end of the fluid cartridge.

13. The syringe of claim 1, wherein the syringe barrel further comprises a cylinder engaged with a rear casing end of the fluid cartridge.

14. The syringe of claim 13, wherein the front member is a helical spring, and whereby in the fluid dispensing pushing the handle will cause the spring to pierce into the sliding member of the fluid cartridge by pushing the cylinder on the rear casing end of the fluid cartridge and holding the fluid cartridge in place.

15. A retractable syringe comprising:
a fluid cartridge including a rear sliding member;
a syringe barrel and hypodermic needle assembly;
a nut member releasably secured to the syringe barrel and hypodermic needle assembly; and
a rear plunger including a rear handle, a front member disposed through the nut member to urge against a rear end of the sliding member, and an arm interconnected the handle and the front member;
wherein in response to dispensing fluid disengage the syringe barrel and hypodermic needle assembly and the fluid cartridge with the nut member and the plunger, and wherein the syringe barrel and hypodermic needle assembly and the fluid cartridge are adapted to discard, and the nut member and the plunger are adapted to sterilize for a next use.

16. The syringe of claim 15, wherein the syringe barrel and hypodermic needle assembly comprises rear outer threads and wherein the nut member comprises front inner threads adapted to secure to the outer threads.

17. The syringe of claim 15, wherein the syringe barrel and hypodermic needle assembly comprises a slip resistant ring engaged with a rear end of the fluid cartridge, and a cylinder engaged with a rear end of the slip resistant ring.

18. The syringe of claim 17, wherein the front member is a helical spring, and whereby in the fluid dispensing pushing the handle will cause the spring to pierce into the sliding member by passing the cylinder and the slip resistant ring.

19. The syringe of claim 15, wherein the syringe barrel and hypodermic needle assembly comprises a cylinder engaged with a rear end of the fluid cartridge.

20. The syringe of claim 19, wherein the front member is a helical spring, and whereby in the fluid dispensing pushing the handle will cause the spring to pierce into the sliding member by passing the cylinder.
